(19) United States
(12) Patent Application Publication
(10) Pub. No.: US 2015/0335837 A1
(43) Pub. Date: Nov. 26, 2015

(54) FLOW SENSORS AND APPARATUS

(71) Applicant: SMITHS MEDICAL INTERNATIONAL LIMITED, Kent (GB)

(72) Inventor: Grant Alan Adams, Coon Rapids, MN (US)

(73) Assignee: SMITHS MEDICAL INTERNATIONAL LIMITED, Kent TN25 4BF (GB)

(21) Appl. No.: 14/758,714

(22) PCT Filed: Dec. 11, 2013
(86) PCT No.: PCT/GB2013/000542

§ 371 Date: Jun. 30, 2015

(30) Foreign Application Priority Data
Jan. 10, 2013 (GB) 1300403.1

(51) Int. Cl. A61M 16/00; A61M 16/08; G01F 1/56

(52) U.S. Cl. CPC A61M 16/0003; G01F 1/56; A61M 16/0066; A61M 16/0875; A61M 2016/003; A61M 2205/3334; A61M 2205/50

(57) ABSTRACT

A gas flow sensor (1), such as for a respiratory tube (120) or a convective warming blanket (40), includes a stiff, flexible rectangular substrate (10) supporting a piezoelectric element (11). The substrate (10) is mounted at its downstream end (15) and aligned in the gas flow (2) so that its free end (16) is vibrated up and down by gas flow. This flexes the substrate (10) and the piezoelectric element (11) so that it provides an alternating output to a processor (20) with an amplitude dependent on the rate of gas flow. The processor (20) provides an output to a display (3) indicative of the gas flow rate.

This invention relates to flow sensors and to apparatus including flow sensors.

The invention is more particularly concerned with gas flow sensors especially, but not exclusively, for medical applications.

There are many different forms of flow sensors such as hot wire anemometers, where an increase in gas flow over the sensor produces an increased cooling effect on the wire, rotating vane devices placed in the gas flow so as to be rotated by the flowing gas at a rate dependent on the gas flow rate, and flexure devices that are placed orthogonally to the flow so that they are deflected to an extent dependent on the pressure exerted by the flowing gas. Examples of previous flow sensors are described, for example in U.S. Pat. No. 7,337,678, U.S. Pat. No. 4,989,456, KR201220135663 and US2012318383. There are many applications in the medical equipment industry where it would be useful to have a low cost gas flow measurement device, such as for control, monitoring or alarm purposes. Presently available gas flow sensors tend to be too expensive to be readily acceptable, especially in single use devices.

It is an object of the present invention to provide an alternative flow sensor.

According to one aspect of the present invention there is provided a flow sensor, characterised in that the sensor includes flexible, elongate piezoelectric member, a support mounting the piezoelectric member generally aligned with the direction of flow in a flow path with one end being supported and located downstream of the opposite end such that the opposite end of the piezoelectric member is free to vibrate in the flow, and that the sensor includes a processor for receiving an electrical output from the piezoelectric member and for providing an output representative of flow dependent on vibration of the piezoelectric member.

The piezoelectric member may be arranged to be vibrated by the fluid flowing along the flow path. The processor may be powered by the output of the piezoelectric member. The processor may include an additional sensor, such as a temperature or pressure sensor, also powered by the output of the piezoelectric member. Alternatively, the piezoelectric member may be driven to vibrate by the processor, the vibration being altered according to the rate of flow of fluid over the sensor. The piezoelectric member may include a stiff, flexible substrate and a piezoelectric element attached to it such that the piezoelectric element is flexed by flexing of the substrate. The piezoelectric element may be of rectangular shape with a blunt edge arranged to face upstream of the flow. The sensor may include two piezoelectric members facing in opposite directions. The processor may provide an output by wireless transmission. The sensor may include a display and the processor may provide the output to the display. The processor may provide the output to a feedback control arranged to control the rate of flow along the flow path such as to maintain a substantially constant rate of flow.

According to another aspect of the present invention there is provided medical ventilation apparatus including a respiratory gas flow tube, characterised in that the apparatus includes a flow sensor according to the above one aspect of the present invention, and that the processor is arranged to provide an output indicative of gas flow along the tube.

According to a further aspect of the present invention there is provided medical temperature management apparatus including a source of air at a controlled temperature and a duct by which air from the source is delivered to the patient, characterised in that the apparatus includes a flow sensor according to the above one aspect of the present invention located in the duct, and that the processor is arranged to provide an output indicative of the rate of air flow along the duct.

The source of air preferably includes a warm air blower and the apparatus may include an inflatable blanket connected with the duct. The output provided by the processor may be arranged to control the source of air so as to maintain a substantially constant flow of air delivered to the patient.

An air flow sensor according to the present invention, and medical apparatus in which it is used, will now be described, by way of example, with reference to the accompanying drawings, in which:

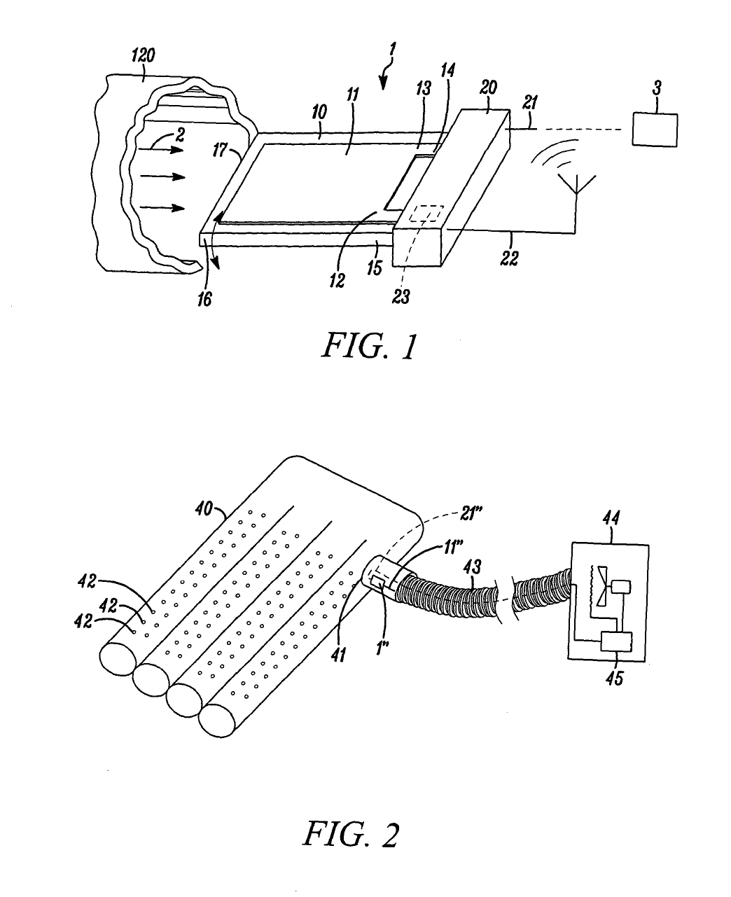

FIG. 1 shows the sensor schematically in a part of medical ventilation apparatus;

FIG. 2 shows the sensor in a convective warming system; and

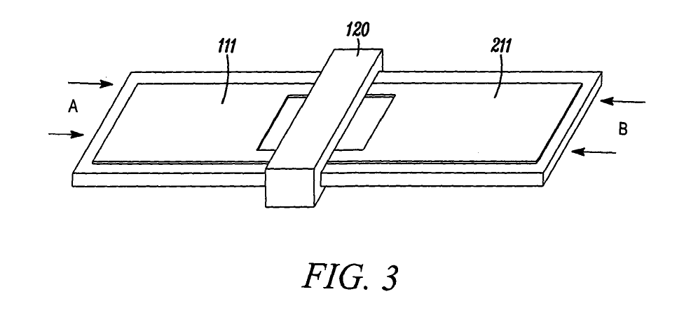

FIG. 3 shows an alternative sensor with two piezoelectric elements facing in opposite directions.

With reference first to FIG. 1 there is shown a flow sensor 1 positioned in a gas flow 2 and providing an output to utilisation means 3, such as a display or control unit. The gas flow 2 may be within a respiratory gas flow tube 120. The flow sensor 1 comprises a stiff but flexible elongate, planar substrate 10, such as of a polycarbonate, and a piezoelectric member in the form of a film element 11 bonded or otherwise attached to the upper surface of the substrate. The piezoelectric film 11 is rectangular in shape and is slightly smaller than the substrate 10, the film being relatively thin so that it is more flexible than the substrate. The film 11 has two terminals 12 and 13 at one edge 14 by which electrical signals are supplied to and from the piezoelectric element. The substrate 10 is mounted at one end 15 with a support in the form of a processing unit 20 so that the length of the substrate extends away from the processing unit and its opposite end 16 is free and unsupported. The free end 16 has a blunt edge 17 facing the gas flow. The edge 14 of the piezoelectric element 11 is located at the supported end 15 of the substrate 10 and the two terminals 12 and 13 make electrical connection with the processing unit 20. The piezoelectric element 11 is arranged so that, when the free end 16 of the substrate 10 is displaced up or down, the piezoelectric element is flexed in a plane at right angles to the plane of the element. This causes the piezoelectric element 11 to be expanded or contracted and thereby produce an alternating output voltage across the two terminals 12 and 13. This output voltage is applied to the processing unit 20.

The substrate 10 with the piezoelectric film 11 and the processing unit 20 is mounted in a gas flow path 2, aligned generally with the direction of gas flow and with the supported end 15 of the substrate 10 being positioned downstream relative to the unsupported end 16. The blunt edge 17 of the substrate 10 and its thickness, material, length and width are selected such that, in combination with the laminated piezoelectric film 11, it will have the desired flexibility so that its free end 16 is caused to flap up and down, or flutter, by flow of gas over the sensor 1. A change in the rate of gas flow will cause a corresponding change in the amplitude of the fluttering and hence also a corresponding change in the output voltage from the piezoelectric element 11. The output voltage is an alternating signal with a frequency equal to the frequency of vibration of the substrate and an amplitude that varies with the amplitude of vibration. The processing unit 20 is preferably powered by the voltage from the piezoelectric element 11 so that the sensor 1 is self powered. The processing unit 20 may include additional sensors 23, which are also powered from the piezoelectric element 11, such as temperature or pressure sensors. The processing unit 20 produces an output signal indicative of gas flow rate and supplies this either via a cable 21, or by a wireless link 22, such as by BlueTooth radio frequency protocol, to the utilisation means 3. The utilisation means 3 may be a display, an alarm that produces a signal when flow rate is outside set limits, a recorder or a feedback control arranged to control the source of the gas flow to maintain a constant level.

In the arrangement described above the piezoelectric element is vibrated by the air or other gas flowing over the element. In an alternative arrangement the piezoelectric element could be driven electrically to vibrate and the processing unit would be arranged to monitor the effect of air flow over the element on vibration of the element. Such an arrangement would require a source of electrical power but could be advantageous in certain situations, such as at low flow rates.

Where the sensor needs to respond to bi-directional gas flow it could be provided with two piezoelectric elements 111 and 211 projecting in opposite directions from a supporting processing unit 120 so that gas flow in one direction “A” causes one of the elements 111 to flutter and gas flow in the opposite direction “B” causes the other element 211 to flutter.

The gas flow sensor can be produced at very low cost making it possible to incorporate it in products where this has not previously been possible, such as in disposable, single-use medical devices. In particular, the sensor could be included in a respiration gas flow tube 120 in medical ventilating apparatus to provide an output indication of gas flow along the tube. A self-powered, wireless sensor could be provided that requires no external electrical connection.

FIG. 2 shows a convective warming arrangement for a patient including a warming blanket 40 such as of the kind sold by Smiths Medical under the Snuggle Warm® trade mark having an air inlet 41 and multiple small air outlet apertures 42 on the side facing the patient. Warm air supplied to the inlet 41 inflates the blanket 40 and flows out steadily from the apertures 42 to maintain the patient's desired body temperature. Warm air is supplied to the blanket 40 via a flexible duct 43 connected at one end to the air inlet 41 and at its opposite end to a warm air blower 44, such as similar to an Equator® blower available from Smiths Medical. An air flow sensor 1″ according to the present invention is mounted in the bore of the duct 43 towards its blanket end. The sensor 1″ is oriented with the free end of the piezoelectric element 11″ facing upstream, towards the blower 44. The sensor 1″ in this arrangement has an electrical cable 21″(although it could be a wireless device) extending from the sensor along the inside of the duct 43 and connected with a modified control unit 45 in the blower 44 so as to provide an alarm function or a feedback function to maintain a set flow rate. Conveniently in this application the flow sensor would incorporate a temperature sensor, of the kind presently used in convective warming arrangements to ensure a correct temperature is maintained at the blanket. Up to now air flow sensors have been too expensive to use in such applications but the sensor of the present invention could be provided at a lower cost.

Although the flow sensor has been described for use in measuring gas flow, it would be possible to use similar sensors to monitor flow of other fluids such as liquids.

1-16. (canceled)

17. A flow sensor, characterized in that the sensor includes a flexible, elongate piezoelectric member, a support mounting the piezoelectric member generally aligned with the direction of flow in a flow path with one end being supported and located downstream of the opposite end such that the opposite end of the piezoelectric member is free to vibrate in the flow, and that the sensor includes a processor for receiving an electrical output from the piezoelectric member and for providing an output representative of flow dependent on vibration of the piezoelectric member.

18. A flow sensor according to claim 17, characterized in that the piezoelectric member is arranged to be vibrated by fluid flowing along the flow path.

19. A flow sensor according to claim 17, characterized in that the processor is powered by the output of the piezoelectric member.

20. A flow sensor according to claim 19, characterized in that the processor includes an additional sensor, such as a temperature or pressure sensor, also powered by the output of the piezoelectric member.

21. A flow sensor according to claim 17, characterized in that the piezoelectric member is driven to vibrate by the processor, and that the vibration is altered according to the rate of flow of fluid over the sensor.

22. A flow sensor according to claim 17, characterized in that the piezoelectric member includes a stiff, flexible substrate and a piezoelectric element attached to it such that the piezoelectric element is flexed by flexing of the substrate.

23. A flow sensor according to claim 17, characterized in that the piezoelectric member is of rectangular shape with a blunt edge arranged to face upstream of the flow.

24. A flow sensor according to claim 17, characterized in that the sensor includes two piezoelectric members facing in opposite directions.

25. A flow sensor according to claim 17, characterized in that the processor provides an output by wireless transmission.

26. A flow sensor according to claim 17, characterized in that the sensor includes a display, and that the processor provides the output to the display.

27. A flow sensor according to claim 17, characterized in that the a processor provides the output to a feedback control arranged to control the rate of flow along the flow path such as to maintain a substantially constant rate of flow.

28. Medical ventilation apparatus including a respiratory gas flow tube, characterized in that the apparatus includes a flow sensor including a flexible, elongate piezoelectric member, a support mounting the piezoelectric member generally aligned with the direction of flow in a flow path with one end being supported and located downstream of the opposite end such that the opposite end of the piezoelectric member is free to vibrate in the flow, and that the sensor includes a processor for receiving an electrical output from the piezoelectric member and for providing an output representative of flow dependent on vibration of the piezoelectric member; and that the processor is arranged to provide an output indicative of gas flow along the tube.

29. Medical temperature management apparatus including a source of air at a controlled temperature and a duct by which air from the source is delivered to the patient, characterized in that the apparatus includes a flow sensor having a flexible, elongate piezoelectric member, a support mounting the piezoelectric member generally aligned with the direction of flow in a flow path with one end being supported and located downstream of the opposite end such that the opposite end of the piezoelectric member is free to vibrate in the flow, and that the sensor includes a processor for receiving an electrical output from the piezoelectric member and for providing an output representative of flow dependent on vibration of the piezoelectric member, that the flow sensor is located in the duct, and that the processor is arranged to provide an output indicative of the rate of air flow along the duct.

30. Medical temperature management apparatus according to claim 29, characterized in that the source of air includes a warm air blower.

31. Medical temperature management apparatus according to claim 29, characterized in that the apparatus includes an inflatable blanket connected with the duct.

32. Medical temperature management apparatus according to claim 29, characterized in that the output provided by the processor is arranged to control the source of air so as to maintain a substantially constant flow of air delivered to the patient.
